SEQUENCE LISTING (12 sequences)

SEQ ID NO: 1 - DNA, 1170 bp, Thermus thermophilus
  CDS (1)..(1170)
atgaagcgcctttccctgagggaggcctggccctacctgaaagacctccagcaagatcccctcgccgtcctgctggcgtggggccgggcccacccccggctcttccttcccctgccccgcttccccctggccctgatctttgaccccgagggggtggagggggcgctcctcgccgaggggaccaccaaggccaccttccagtaccgggccctctcccgcctcacggggaggggcctcctcaccgactggggggaaagctggaaggaggcgcgcaaggccctcaaagaccccttcctgccgaagaacgtccgcggctaccgggaggccatggaggaggaggcccgggccttcttcggggagtggcggggggaggagcgggacctggaccacgagatgctcgccctctccctgcgcctcctcgggcgggccctcttcgggaagcccctctccccaagcctcgcggagcacgcccttaaggccctggaccggatcatggcccagaccaggagccccctggccctcctggacctggccgccgaagcccgcttccggaaggaccggggggccctctaccgcgaggcggaagccctcatcgtccacccgcccctctcccaccttccccgagagcgcgccctgagcgaggccgtgaccctcctggtggcgggccacgagacggtggcgagcgccctcacctggtcctttctcctcctctcccaccgcccggactggcagaagcgggtggccgagagcgaggaggcggccctcgccgccttccaggaggccctgaggctctacccccccgcctggatcctcacccggaggctggaaaggcccctcctcctgggagaggaccggctccccccgggcaccaccctggtcctctccccctacgtgacccagaggctccacttccccgatggggaggccttccggcccgagcgcttcctggaggaaagggggaccccttcggggcgctacttcccctttggcctggggcagaggctctgcctggggcgggacttcgccctcctcgagggccccatcgtcctcagggccttcttccgccgcttccgcctagaccccctccccttcccccgggtcctcgcccaggtcaccctgaggcccgaaggcgggcttcccgcgcggcctagggaggaggtgcgggcgtga

SEQ ID NO: 2 - protein, 389 aa, Thermus thermophilus
MKRLSLREAWPYLKDLQQDPLAVLLAWGRAHPRLFLPLPRFPLALIFDPEGVEGALLAEGTTKATFQYRALSRLTGRGLLTDWGESWKEARKALKDPFLPKNVRGYREAMEEEARAFFGEWRGEERDLDHEMLALSLRLLGRALFGKPLSPSLAEHALKALDRIMAQTRSPLALLDLAAEARFRKDRGALYREAEALIVHPPLSHLPRERALSEAVTLLVAGHETVASALTWSFLLLSHRPDWQKRVAESEEAALAAFQEALRLYPPAWILTRRLERPLLLGEDRLPPGTTLVLSPYVTQRLHFPDGEAFRPERFLEERGTPSGRYFPFGLGQRLCLGRDFALLEGPIVLRAFFRRFRLDPLPFPRVLAQVTLRPEGGLPARPREEVRA

SEQ ID NO: 3 - DNA, 1188 bp, Artificial sequence
  misc_feature (4)..(21): His tag
atgcatcaccatcatcatcacaagcgcctttccctgagggaggcctggccctacctgaaagacctccagcaagatcccctcgccgtcctgctggcgtggggccgggcccacccccggctcttccttcccctgccccgcttccccctggccctgatctttgaccccgagggggtggagggggcgctcctcgccgaggggaccaccaaggccaccttccagtaccgggccctctcccgcctcacggggaggggcctcctcaccgactggggggaaagctggaaggaggcgcgcaaggccctcaaagaccccttcctgccgaagaacgtccgcggctaccgggaggccatggaggaggaggcccgggccttcttcggggagtggcggggggaggagcgggacctggaccacgagatgctcgccctctccctgcgcctcctcgggcgggccctcttcgggaagcccctctccccaagcctcgcggagcacgcccttaaggccctggaccggatcatggcccagaccaggagccccctggccctcctggacctggccgccgaagcccgcttccggaaggaccggggggccctctaccgcgaggcggaagccctcatcgtccacccgcccctctcccaccttccccgagagcgcgccctgagcgaggccgtgaccctcctggtggcgggccacgagacggtggcgagcgccctcacctggtcctttctcctcctctcccaccgcccggactggcagaagcgggtggccgagagcgaggaggcggccctcgccgccttccaggaggccctgaggctctacccccccgcctggatcctcacccggaggctggaaaggcccctcctcctgggagaggaccggctccccccgggcaccaccctggtcctctccccctacgtgacccagaggctccacttccccgatggggaggccttccggcccgagcgcttcctggaggaaagggggaccccttcggggcgctacttcccctttggcctggggcagaggctctgcctggggcgggacttcgccctcctcgagggccccatcgtcctcagggccttcttccgccgcttccgcctagaccccctccccttcccccgggtcctcgcccaggtcaccctgaggcccgaaggcgggcttcccgcgcggcctagggaggaggtgcgggcgtga

SEQ ID NO: 4 - protein, 395 aa, Artificial sequence
  Description of the artificial sequence N-terminal his tagged
MHHHHHHKRLSLREAWPYLKDLQQDPLAVLLAWGRAHPRLFLPLPRFPLALIFDPEGVEGALLAEGTTKATFQYRALSRLTGRGLLTDWGESWKEARKALKDPFLPKNVRGYREAMEEEARAFFGEWRGEERDLDHEMLALSLRLLGRALFGKPLSPSLAEHALKALDRIMAQTRSPLALLDLAAEARFRKDRGALYREAEALIVHPPLSHLPRERALSEAVTLLVAGHETVASALTWSFLLLSHRPDWQKRVAESEEAALAAFQEALRLYPPAWILTRRLERPLLLGEDRLPPGTTLVLSPYVTQRLHFPDGEAFRPERFLEERGTPSGRYFPFGLGQRLCLGRDFALLEGPIVLRAFFRRFRLDPLPFPRVLAQVTLRPEGGLPARPREEVRA

SEQ ID NO: 5 - DNA, 1188 bp, Artificial sequence
  misc_feature (1168)..(1185): His tag
atgaagcgcctttccctgagggaggcctggccctacctgaaagacctccagcaagatcccctcgccgtcctgctggcgtggggccgggcccacccccggctcttccttcccctgccccgcttccccctggccctgatctttgaccccgagggggtggagggggcgctcctcgccgaggggaccaccaaggccaccttccagtaccgggccctctcccgcctcacggggaggggcctcctcaccgactggggggaaagctggaaggaggcgcgcaaggccctcaaagaccccttcctgccgaagaacgtccgcggctaccgggaggccatggaggaggaggcccgggccttcttcggggagtggcggggggaggagcgggacctggaccacgagatgctcgccctctccctgcgcctcctcgggcgggccctcttcgggaagcccctctccccaagcctcgcggagcacgcccttaaggccctggaccggatcatggcccagaccaggagccccctggccctcctggacctggccgccgaagcccgcttccggaaggaccggggggccctctaccgcgaggcggaagccctcatcgtccacccgcccctctcccaccttccccgagagcgcgccctgagcgaggccgtgaccctcctggtggcgggccacgagacggtggcgagcgccctcacctggtcctttctcctcctctcccaccgcccggactggcagaagcgggtggccgagagcgaggaggcggccctcgccgccttccaggaggccctgaggctctacccccccgcctggatcctcacccggaggctggaaaggcccctcctcctgggagaggaccggctccccccgggcaccaccctggtcctctccccctacgtgacccagaggctccacttccccgatggggaggccttccggcccgagcgcttcctggaggaaagggggaccccttcggggcgctacttcccctttggcctggggcagaggctctgcctggggcgggacttcgccctcctcgagggccccatcgtcctcagggccttcttccgccgcttccgcctagaccccctccccttcccccgggtcctcgcccaggtcaccctgaggcccgaaggcgggcttcccgcgcggcctagggaggaggtgcgggcgcatcaccatcatcatcactga

SEQ ID NO: 6 - protein, 395 aa, Artificial sequence
  Description of the artificial sequence C-terminal His-tagged
MKRLSLREAWPYLKDLQQDPLAVLLAWGRAHPRLFLPLPRFPLALIFDPEGVEGALLAEGTTKATFQYRALSRLTGRGLLTDWGESWKEARKALKDPFLPKNVRGYREAMEEEARAFFGEWRGEERDLDHEMLALSLRLLGRALFGKPLSPSLAEHALKALDRIMAQTRSPLALLDLAAEARFRKDRGALYREAEALIVHPPLSHLPRERALSEAVTLLVAGHETVASALTWSFLLLSHRPDWQKRVAESEEAALAAFQEALRLYPPAWILTRRLERPLLLGEDRLPPGTTLVLSPYVTQRLHFPDGEAFRPERFLEERGTPSGRYFPFGLGQRLCLGRDFALLEGPIVLRAFFRRFRLDPLPFPRVLAQVTLRPEGGLPARPREEVRAHHHHHH

SEQ ID NO: 7 - DNA, 30 bp, Artificial sequence
  Description of the artificial sequence PCR primer
cgaagctcatatgaagcgcctttccctgag

SEQ ID NO: 8 - DNA, 30 bp, Artificial sequence
  Description of the artificial sequence PCR primer
gcgaattcacgcccgcacctcctccctagg

SEQ ID NO: 9 - DNA, 42 bp, Artificial sequence
  Description of the artificial sequence PCR primer
cgaagctcatatgcatcaccatcatcatcacaagcgcctttc

SEQ ID NO: 10 - DNA, 42 bp, Artificial sequence
  Description of the artificial sequence PCR primer
cggaattcagtgatgatgatggtgatgcgcccgcacctcctc

SEQ ID NO: 11 - DNA, 30 bp, Artificial sequence
  Description of the artificial sequence PCR primer
ccggaattcatgaagcgcctttccctgagg

SEQ ID NO: 12 - DNA, 44 bp, Artificial sequence
  Description of the artificial sequence PCR primer
ccaatgcattggttctgcagtcaggcccgcacctcctccctagg

(19) United States
(12) Patent Application Publication
(10) Pub. No.: US 2006/0063226 A1
(43) Pub. Date: Mar. 23, 2006

(54) Method for the biotransformation of carotenoids by means of a cytochrome p450 monooxygnase

(75) Inventors: Markus Matuschek, Weinheim (DE); Bernhard Hauer, Fussgonheim (DE); Rolf Schmid, Stuttgart (DE); Isabelle Kauffmann, Stuttgart (DE); Francesca Blasco, Esslingen (DE); Claudia Schmidt-Dannert, Shoreview, MN (US)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 10/521,916

(22) PCT Filed: Jul. 25, 2003
(86) PCT No.: PCT/EP03/08199

§ 371 Date: Oct. 31, 2005

(30) Foreign Application Priority Data
Jul. 26, 2002 (DE) 102 34 126.5

(51) Int. Cl. C12P 23/00; A61K 31/12; A61K 31/336

(52) U.S. Cl. 435/67; 514/690; 514/475

(57) ABSTRACT

The invention relates to a process for the biotransformation of carotenoids using enzymes having cytochrome P450 monooxygenase activity; in particular monooxygenases from thermophilic bacteria, in particular of the genus Thermus sp.

The invention relates to a process for the biotransformation of carotenoids using enzymes having cytochrome P450 monooxygenase activity; in particular monooxygenases from thermophilic bacteria, in particular of the genus Thermus sp., and to the microorganisms and expression constructs which can be used for such processes.

### PRIOR ART

Xanthophylls such as zeaxanthin and cryptoxanthin are oxygen-containing carotenoids and represent, as pigments or precursors for vitamin A derivatives, important additives to human or animal diet. Xanthophylls are also attributed with a health-promoting effect. They strengthen the immune response and, because of their antioxidant properties, have a cancer-preventive effect, which makes them of interest as nutraceuticals.

Cytochrome P450 monooxygenases have the ability to catalyze oxygenation reactions of industrial interest and have therefore been intensively investigated for some time. Thus, for example, the cytochrome P450 monooxygenase BM-3 from Bacillus megaterium has been isolated and characterized and is now available by the recombinant route (cf., for example, DE-A-199 35 115).

This cytochrome P450 monooxygenase normally catalyzes the subterminal hydroxylation of long-chain saturated acids and of the corresponding amides and alcohols thereof or the epoxidation of unsaturated long-chain fatty acids or saturated fatty acids with a medium chain length. The optimal chain length of saturated fatty acids is 14 to 16 carbon atoms.

The structure of the heme domain of P450 BM-3 has been determined by x-ray structure analysis. The substrate binding site is in the form of a long tunnel-like orifice which extends from the surface of the molecule to the heme molecule and is bounded almost exclusively by hydrophobic amino acid residues. The only charged residues on the surface of the heme domain are the residues Arg47 and Tyr51. It is assumed that these are involved in the binding of the carboxylate group of the substrate through formation of a hydrogen bond. It has now become possible, by targeted introduction of point mutations, to extend the substrate range of this enzyme. Thus, it is now possible for shorter- and longer-chain carboxylic acids, alkanes, alkenes, cycloalkanes, cycloalkenes and diverse aromatic compounds also to be oxidized by this enzyme (cf. DE-A-199 35 115, 199 55 605, 100 11 723 and 100 14 085).

WO-A-02/33057 discloses cytochrome P450 monooxygenases from thermophilic bacteria which are suitable for the biotransformation of various organic substrates. Carotenoids such as, for example, β-carotene are not mentioned therein as potential substrate of the cytochrome P450 monooxygenases.

DE-A-199 16 140 describes a carotene hydroxylase from the green alga Haematococcus pluvialis which catalyzes inter alia the conversion of β-carotene into zeaxanthin and cryptoxanthin. There is no reference to the possible utility of cytochrome P450 monooxygenases in the biotransformation of β-carotene.

In order to improve further the industrial utilizability of the class of cytochrome P450 monooxygenase enzymes, it would therefore be desirable to find novel areas of application thereof.

### BRIEF DESCRIPTION OF THE INVENTION

It is an object of the present invention to provide novel areas of application of cytochrome P450 monooxygenases.

We have found that this object is achieved by providing a process for the oxidation of carotenoids, which comprises reacting a carotenoid in the presence of an enzyme having cytochrome P450 monooxygenase activity, which is additionally capable of carotenoid oxidation, and isolating the oxidation product.

An enzyme having cytochrome P450 monooxygenase activity and which is additionally capable of carotenoid oxidation has the effect according to the invention of introducing a hydroxyl group on the carbon in position 3 of a β-ionone ring or on the carbon in position 3 of a 4-keto-β-ionone ring.

Examples of suitable carotenoids are β,β-carotene (referred to as β-carotene hereinafter), β,ε-carotene or canthaxanthin.

A carotene oxidation within the meaning of the invention comprises mono- or polyhydroxylation of the carotene.

Oxidation products resulting according to the invention preferably comprise zeaxanthin, cryptoxanthin, adonirubin, astaxanthin, lutein or mixtures thereof.

### DETAILED DESCRIPTION

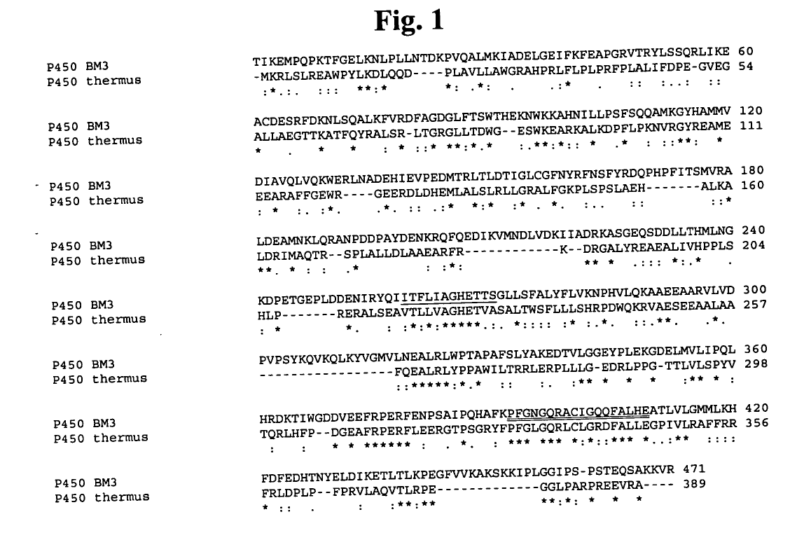

The invention is now explained in detail with reference to the appended figures. These show FIG. 1 a sequence comparison of P450 from Thermus thermophilus with the heme domain of P450 BM3 from Bacillus megaterium. The heme binding site is shown double-underlined (Cys400 in P450 BM3 is the cysteine residue which coordinates with the iron atom of the prosthetic group). The region in contact with the T-end of the fatty acid chain is singly underlined. The extent of agreement is indicated by various symbols (“*”=identical residues; “:” and “.”=similar residues).

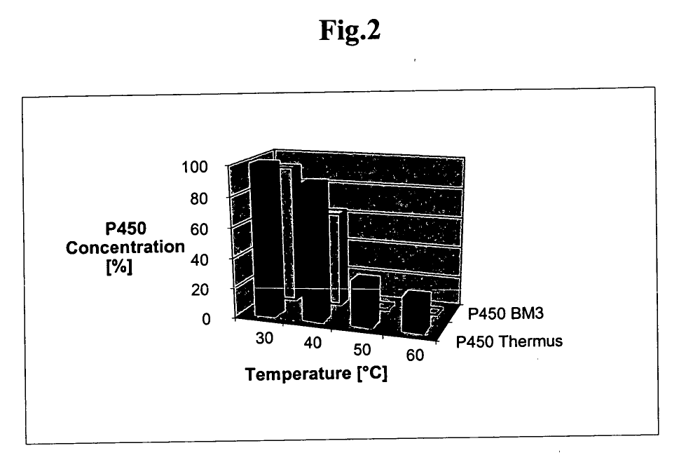

FIG. 2 shows the result of a comparative test to determine the thermal stability of P450 BM3 and P450 from Thermus sp. The thermal stability was determined via the heme group content by spectrometry in the wavelength range between 400 and 500 nm.

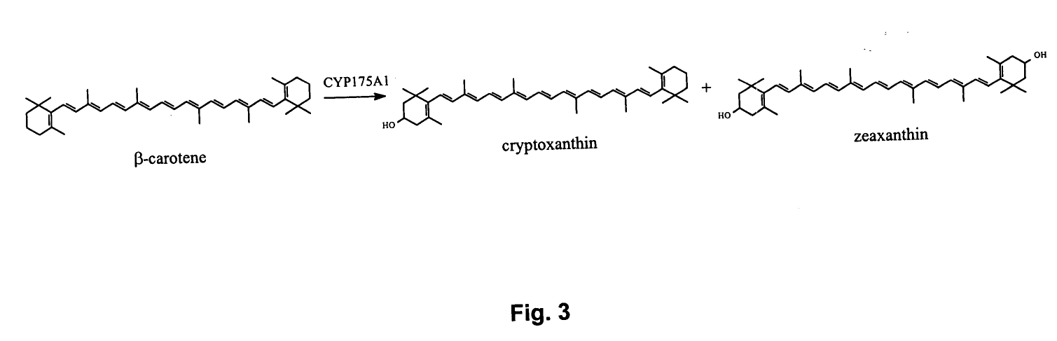

FIG. 3 shows a reaction scheme for the biotransformation according to the invention of β-carotene to cryptoxanthin and zeaxanthin.

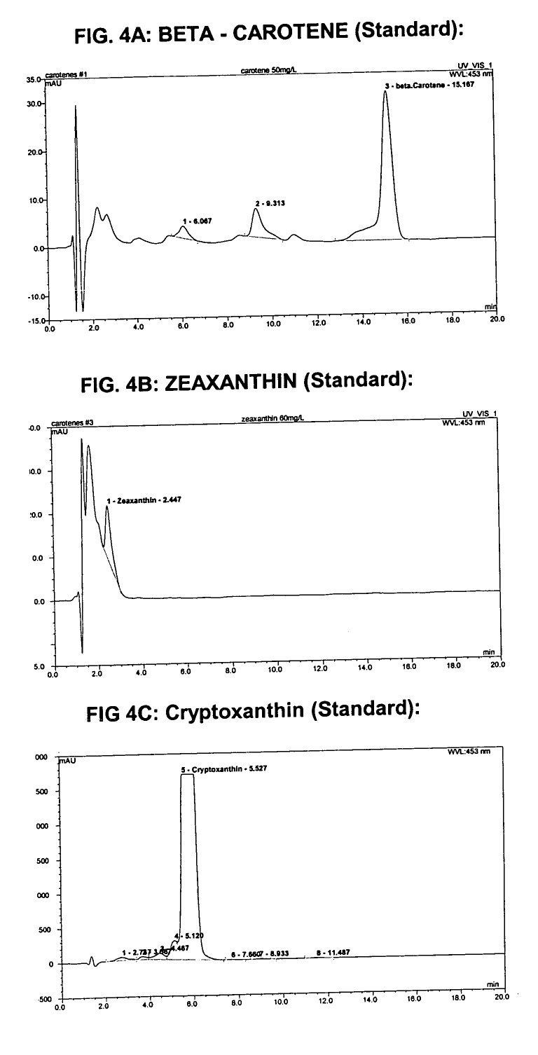

FIG. 4 shows the HPLC elution profile of standard samples containing β-carotene, zeaxanthin and cryptoxanthin.

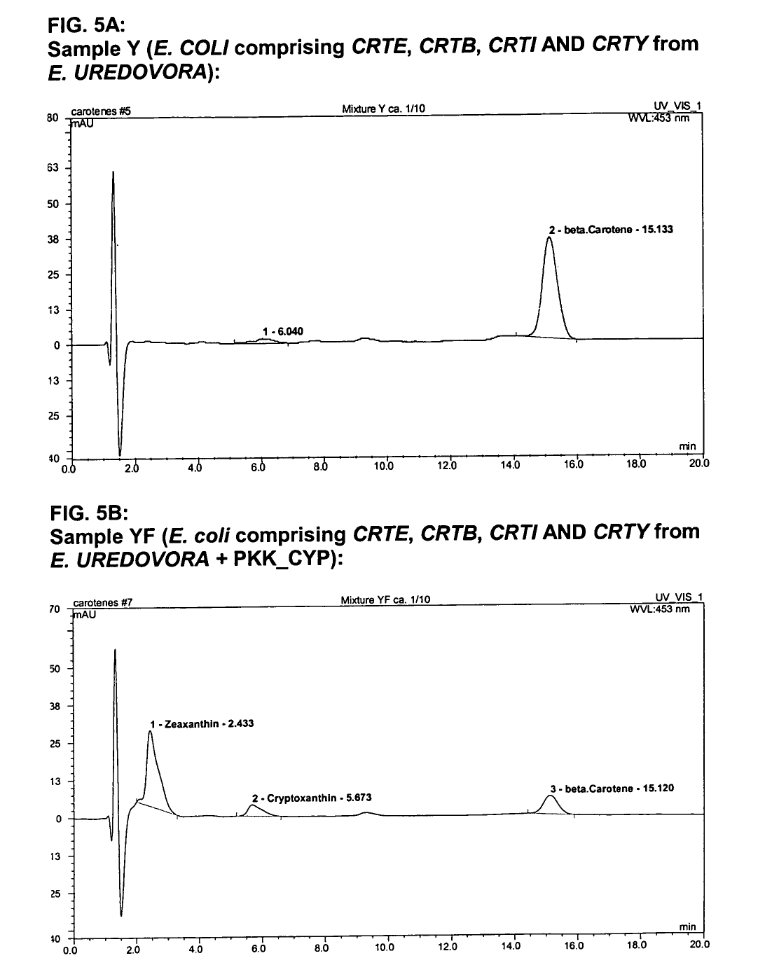

FIG. 5 illustrates the results of biotransformation experiments with recombinant E. coli strains which, besides the carotenogenic genes crtE, crtB, crtI and crtY (FIG. 5A), are transformed with a construct pKK_CYP according to the invention (FIG. 5B); a significant production of zeaxanthin and cryptoxanthin is observed in the presence of pKK_CYP.

### A) PROCESS FOR CARATENOID OXIDATION

A first aspect of the invention relates in particular to a process for oxidizing carotenoids, such as, for example, β-carotene, where
a1) a recombinant microorganism which produces an enzyme having cytochrome P450 monooxygenase activity is cultivated in a culture medium in the presence of exogenous carotenoid or carotenoid formed as intermediate; or
a2) a carotenoid-containing reaction medium is incubated with an enzyme having cytochrome P450 monooxygenase activity; and
b) the oxidation product formed or a secondary product thereof is isolated from the medium.

The process of the invention is carried out under conditions which preferably promote, but at least do not impede or even inhibit, the oxidation of carotenoids such as β-carotene. The oxidation preferably takes place by cultivating the recombinant microorganism in the presence of oxygen at a cultivation temperature of at least about 20° C., such as, for example, 20 to 40° C., and at a pH of about 6 to 9.

The microorganisms preferably used are those able through heterologous complementation to produce carotenoids, such as, for example, to produce β-carotene, and additionally express an enzyme having cytochrome P450 monooxygenase activity. E. coli strains with heterologous complementation, and other microorganisms into which a P450 monooxygenase activity according to the invention (with carotenoid-oxidizing activity) can be incorporated in an analogous way, are described, for example, in the abovementioned DE-A-199 16 140, which is incorporated herein by reference.

In another preferred variant, carotenoid such as, for example, β-carotene is added as exogenous substrate to a medium, and the oxidation is carried out by enzymatic reaction of the substrate-containing medium in the presence of oxygen at a temperature of at least about 20° C. and at a pH of about 6 to 9, it being possible for the substrate-containing medium additionally to comprise an approximately 10- to 100-fold molar excess, based on the substrate, of reducing equivalents.

The above processes can preferably be carried out in bioreactors. The invention therefore relates to such bioreactors comprising at least one monooxygenase of the invention or at least one recombinant microorganism of the invention, where appropriate in immobilized form in each case.

If the reaction is carried out with a recombinant microorganism, the cultivation of the microorganisms preferably takes place initially in the presence of oxygen and in a complex medium such as, for example, TB or LB medium at a cultivation temperature of about 20° C. or more, and at a pH of about 6 to 9, until an adequate cell density is reached. In order to be able to control the oxidation reaction better, it is preferred to use an inducible promoter. The cultivation is continued after induction of the monooxygenase production in the presence of oxygen for 12 hours to 3 days, for example.

If, on the other hand, the reaction according to the invention is carried out with purified or enriched enzyme, the enzyme of the invention is dissolved or solubilized in a medium containing exogenous substrate (about 0.01 to 10 mM, or 0.05 to 5 mM), and the reaction is preferably carried out in the presence of oxygen at a temperature of about 10° C. or more, and at a pH of about 6 to 9 (as adjusted for example with 100 to 200 mM phosphate or Tris buffer), and in the presence of a reducing agent, with the substrate-containing medium additionally containing an approximately 10- to 100-fold molar excess of reducing equivalents (electron donor) based on the substrate to be oxidized. The preferred reducing agent is NADPH.

In the substrate oxidation process of the invention there is reductive enzymatic cleavage of oxygen which is present in or added to the reaction medium. The necessary reducing equivalents are made available by the added reducing agent (electron donor).

The oxidation product formed can then be removed from the medium and purified in a conventional way, such as, for example, by extraction and/or chromatography. Suitable methods are known to the skilled worker and therefore require no special explanation.

Particularly preferred processes are those in which the cytochrome P450 monooxygenase employed has an amino acid sequence which comprises a part-sequence from amino acid residue Pro328 to Glu345 as shown in SEQ ID NO:2; and, where appropriate, additionally a part-sequence from amino acid residue Val216 to Ala227 as shown in SEQ ID NO:2.

Particularly preferred processes are those using a monooxygenase which has an amino acid sequence which comprises at least one other part-sequence which is selected from part-sequences of at least 10 consecutive amino acids from the sequence regions specified by the amino acid residues Met1 to Phe327 and Gly346 to Ala389 as shown in SEQ ID NO:2; and, in particular, those processes using a monooxygenase which has an amino acid sequence which essentially corresponds to SEQ ID NO:2.

Carrying out the process of the invention with the aid of microorganisms entails cultivation of a recombinant microorganism which harbors an expression construct which comprises, under the control of regulatory nucleotide sequences, the coding sequence for a cytochrome P450 monooxygenase as defined above.

Another aspect of the invention relates to the use of a cytochrome P450 monooxygenase as defined above or of a nucleotide sequence coding therefore for the microbiological oxidation of carotenoids such as, for example, β-carotene.

### B) RECOMBINANT MICROORGANISMS FOR CARRYING OUT THE PROCESS

The invention additionally relates to recombinant microorganisms which are able through heterologous complementation to produce carotenoids, such as, for example, to produce β-carotene, and additionally express an enzyme having cytochrome P450 monooxygenase activity. The heterologous complementation of such microorganisms is preferably with carotenogenic genes such as, for example, crtE, crtB, crtI and crtY. They are derived in particular from bacteria of the genus Escherichia sp, such as E. coli, in particular E. coli JM 109.

Microorganisms of the invention are transformed in particular with an expression vector which comprises, under the genetic control of regulatory nucleotide sequences, the coding sequence for a cytochrome P450 monooxygenase as defined above.

A preferred expression vector comprising the coding sequence for a cytochrome P450 monooxygenase as defined above comprises upstream thereof the strong tac promoter and downstream the strong rrnB ribosomal terminator in operative linkage.

Further microorganisms which can be used and their production for carrying out the process of the invention are disclosed, for example, in DE-A-199 16 140, which is incorporated herein by reference.

The invention also relates to the use of the P450 enzymes having carotenoid-, in particular β-carotene-, oxidizing activity of the invention or of the encoding nucleic acid sequence thereof for producing genetically modified organisms, in particular for carrying out the process of the invention.

The invention further relates to organisms which have been correspondingly genetically modified, where expression of the gene for the carotenoid-, in particular β-carotene-, oxidizing activity of the invention is increased by comparison with a wild type in the case where the starting organism contains the gene used according to the invention, or is caused in the case where the starting organism does not contain the gene used according to the invention, by the genetic modification.

A genetically modified organism means an organism in which the P450 genes or nucleic acid constructs of the invention have been inserted, preferably by one of the methods described herein.

The genetically modified organism comprises at least one carotenoid-, in particular β-carotene-, oxidizing gene of the invention or at least one nucleic acid construct of the invention. Depending on the starting organism, the nucleic acid may be present in the chromosome or outside the chromosome.

The genetically modified organisms preferably display altered carotenoid metabolism compared with the wild type.

Suitable genetically modified organisms are in principle all organisms able to synthesize carotenoids or xanthophylls.

Preferred starting organisms are those naturally able to synthesize xanthophylls. However, starting organisms which are able to synthesize xanthophylls owing to the introduction of genes of carotenoid biosynthesis are also suitable.

Starting organisms mean prokaryotic or eukaryotic organisms such as, for example, microorganisms or plants. Preferred microorganisms are bacteria, yeasts, algae or fungi.

Bacteria which can be used are both bacteria which are able, because of the introduction of carotenoid biosynthesis genes of a carotenoid-producing organism, to synthesize xanthophylls, such as, for example, bacteria of the genus Escherichia which contain, for example, crt genes from Erwinia, and bacteria which are intrinsically able to synthesize xanthophylls, such as, for example, bacteria of the genus Erwinia, Agrobacterium, Flavobacterium, Alcaligenes or cyanobacteria of the genus Synechocystis. Preferred bacteria are Escherichia coli, Erwinia herbicola, Erwinia uredovora, Agrobacterium aurantiacum, Alcaligenes sp. PC-1, Flavobacterium sp. strain R1534, the cyanobacterium Synechocystis sp. PCC6803, Paracoccus marcusu, or Paracoccus carotinifaciens.

Preferred yeasts are Candida, Saccharomyces, Hansenula or Pichia.

Preferred fungi are Aspergillus, Trichoderma, Ashbya, Neurospora, Blakeslea, Phycomyces, Fusarium or other fungi described in Indian Chem. Engr. Section B. Vol. 37, No. 1, 2 (1995) on page 15, Table 6.

Preferred algae are green algae such as, for example, algae of the genus Haematococcus, Phaedactylum tricornatum, Volvox or Dunaliella. Particularly preferred algae are Haematococcus pluvialis or Dunaliella bardawil.

In a preferred embodiment, plants are used as starting organisms and, accordingly, also as genetically modified organisms. Examples of preferred plants are tagetes, sunflower, arabidopsis, tobacco, red pepper, soybean, tomato, aubergine, paprika, carrot, potato, corn, lettuce and brassica species, oats, rye, wheat, triticale, millet, rice, alfalfa, flax, brassicaceae such as, for example, oilseed rape or canola, sugar beet, sugar cane or woody plants such as, for example, aspen or yew.

Particular preference is given to Arabidopsis thaliana, Tagetes erecta, oilseed rape, canola, potatoes and oil seeds and typical carotenoid producers such as soybean, sunflower, paprika, carrot, pepper or corn.

### C) ENZYMES, POLYNUCLEOTIDES AND CONSTRUCTS

Cytochrome P450 monooxygenases which can be used according to the invention can be isolated in particular from thermophilic bacteria, preferably of the genus Thermus sp., such as, for example, of the species Thermus thermophilus, strain HB27 (deposited at the DSM under the number DSM7039). “Thermophilic” bacteria meet according to the invention the temperature tolerance criteria of H. G. Schlegel, Allgemeine Mikrobiologie, Thieme Verlag Stuttgart, 5th edition, page 173, for thermophilic and extremely thermophilic organisms (i.e. growth optimum at above 40° C.).

The monooxygenases preferably used according to the invention are preferably characterized by increased thermal stability. This is manifested by a loss of activity at elevated temperature (e.g. in a range from 30 to 60° C., pH 7.5, 25 mM Tris/HCl) which is less than that of the P450 BM-3 from Bacillus megaterium.

In a preferred embodiment, a cytochrome P450 monooxygenase from the thermophilic bacterium T. thermophilus is used according to the invention. The protein has a molecular weight of about 44 kDa (determined by SDS gel electrophoresis), is soluble and shows in the reduced state, oxidized state and as carbonyl adduct an absorption spectrum analogous to that of other P450 enzymes. Comparisons of the sequences of this T. thermophilus enzyme of the invention and other known P450 enzymes established the following identities: P450 BM3, 32% identity; CYP119, 29% identity; P450eryF, 31% identity. The enzyme of the invention shows exceptional thermal stability, illustrated by a melting temperature of about 85° C., which is about 30° C. above the value for P450cam.

A further aspect of the invention relates to the use of polynucleotides which code for a cytochrome P450 monooxygenase, in particular a cytochrome P450 monooxygenase from the genus Thermus sp., in processes for oxidizing β-carotene.

Preferred polynucleotides are those essentially having a nucleic acid sequence as shown in SEQ ID NO: 1, and the nucleic acid sequences complementary thereto and derived therefrom.

A further aspect of the invention relates to the use of expression cassettes or of recombinant vectors for producing recombinant microorganisms which can be used for the reactions of the invention.

The invention likewise encompasses the use of “functional equivalents” of the specifically disclosed novel P450 monooxygenases for the reactions of the invention.

“Functional equivalents” or analogs of the specifically disclosed monooxygenases are, for the purposes of the present invention, enzymes which are different therefrom but which still have the desired substrate specificity within the scope of the oxidation reaction identified above and/or have increased thermal stability compared with P450 BM3, e.g. at temperatures in the range from about 30 to 60° C. and, where appropriate, higher temperatures after treatment in 25 mM Tris/HCl for 30 minutes.

“Functional equivalents” mean according to the invention in particular mutants which have in at least one of the abovementioned sequence positions an amino acid which differs from that specifically mentioned but nevertheless catalyze one of the abovementioned oxidation reactions. “Functional equivalents” thus comprise the mutants obtainable by one or more, such as, for example, 1 to 30 or 1 to 20 or 1 to 10, amino acid additions, substitutions, deletions and/or inversions, it being possible for said modifications to occur in any sequence position as long as they lead to a mutant having the profile of properties of the invention. Functional equivalence exists in particular also when there is qualitative agreement between mutant and unmodified enzyme in the reactivity pattern, i.e. there are differences in the rate of conversion of identical substrates, for example.

“Functional equivalents” included according to the invention have an amino acid sequence which differs from SEQ ID NO: 2 in at least one position, with the modification in the sequence preferably altering the monooxygenase activity only inconsiderably, that is to say by not more than about ±90%, in particular ±50% or not more than ±30%. This alteration can be determined by using a reference substrate such as, for example, β-carotene under standardized conditions (for example 0.1 to 0.5 M substrate, pH range 6 to 8, in particular 7; T=30 to 70° C.).

“Functional equivalents” in the above sense are also precursors of the described polypeptides, and functional derivatives and salts of the polypeptides. The term “salts” means both salts of carboxyl groups and acid addition salts of amino groups of the protein molecules of the invention. Salts of carboxyl groups can be prepared in a manner known per se and comprise inorganic salts such as, for example, sodium, calcium, ammonium, iron and zinc salts, and salts with organic bases such as, for example amines, such as triethanolamine, arginine, lysine, piperidine and the like. Acid addition salts, such as, for example, salts with mineral acids, such as hydrochloric acid or sulfuric acid, and salts with organic acids such as acetic acid and oxalic acid are likewise an aspect of the invention.

“Functional derivatives” of polypeptides of the invention can likewise be prepared on functional amino acid side groups or on the N- or C-terminal end thereof by known techniques. Derivatives of this type comprise, for example, aliphatic esters of carboxyl groups, amides of carboxyl groups, obtainable by reaction with ammonia or with a primary or secondary amine; N-acyl derivatives of free amino groups prepared by reaction with acyl groups; or O-acyl derivatives of free hydroxyl groups prepared by reaction with acyl groups.

“Functional equivalents” included according to the invention are homologs of the specifically disclosed proteins. These have a homology of at least 60%, preferably at least 75%, in particular at least 85%, such as, for example, 90%, 95% or 99%, with one of the specifically disclosed sequences, calculated by the algorithm of Pearson and Lipman, Proc. Natl. Acad. Sci. (USA) 85(8), 1988, 2444-2448.

Homologs of the proteins or polypeptides of the invention can be generated by mutagenesis, e.g. by point mutation or truncation of the protein.

Homologs of the proteins of the invention can be identified by screening combinatorial libraries of mutants such as, for example, truncation mutants. It is possible, for example, to generate a variegated library of protein variants by combinatorial mutagenesis at the nucleic acid level, such as, for example, by enzymatic ligation of a mixture of synthetic oligonucleotides. There is a large number of methods which can be used to produce libraries of potential homologs from a degenerate oligonucleotide sequence. Chemical synthesis of a degenerate gene sequence can be carried out in an automatic DNA synthesizer, and the synthetic gene can then be ligated into a suitable expression vector. The use of a degenerate set of genes makes it possible to provide all sequences which encode the desired set of potential protein sequences in one mixture. Processes for synthesizing degenerate oiigonucleotides are known to the skilled worker (for example Narang, S. A. (1983) Tetrahedron 39:3; Itakura et al. (1984) Annu. Rev. Biochem. 53:323; Itakura et al. (1984) Science 198:1056; Ike et al. (1983) Nucleic Acids Res. 11:477).

“Functional equivalents” naturally also encompass P450 monooxygenases which are obtainable from other organisms, e.g. from bacteria other than those specifically mentioned herein, and naturally occurring variants. For example, homologous sequence regions can be found by sequence comparison, and equivalent enzymes can be established on the basis of the specific requirements of the invention.

The invention also relates to the use of nucleic acid sequences (single- and double-stranded DNA and RNA sequences) coding for one of the above monooxygenases and their functional equivalents for carrying out the above processes. Further nucleic acid sequences of the invention are derived from SEQ ID NO: 1 and differ therefrom through addition, substitution, insertion or deletion of one or more nucleotides, but still code for a monooxygenase having the desired profile of properties.

All the nucleic acid sequences mentioned herein can be prepared in a manner known per se by chemical synthesis from the nucleotide building blocks, such as, for example, by fragment condensation of individual overlapping, complementary nucleic acid building blocks of the double helix. The chemical synthesis of oligonucleotides can take place, for example, in a known manner by the phosphoramidite method (Voet, Voet, Biochemie, 2nd edition, Wiley Press New York, pages 896-897). The annealing of synthetic oligonucleotides and filling in of gaps using the Klenow fragment of DNA polymerase and ligation reactions, and general cloning methods, are described in Sambrook et al. (1989), Molecular Cloning: A laboratory manual, Cold Spring Harbor Laboratory Press.

The invention also encompasses nucleic acid sequences which comprise so-called silent mutations or are modified, by comparison with a specifically mentioned sequence, in accordance with the codon usage of a specific source or host organism, as well as naturally occurring variants, such as, for example, splice variants thereof. It likewise relates to sequences which are obtainable by conservative nucleotide substitutions (i.e. the relevant amino acid is replaced by an amino acid of the same charge, size, polarity and/or solubility).

The invention additionally encompasses nucleic acid sequences which hybridize with or are complementary to the abovementioned coding sequences. These polynucleotides can be found by scanning genomic or cDNA libraries and, where appropriate, be amplified therefrom by means of PCR using suitable primers, and then, for example, be isolated with suitable probes. Another possibility is to transform suitable microorganisms with polynucleotides or vectors of the invention, multiply the microorganisms and thus the polynucleotides, and then isolate them. An additional possibility is to synthesize polynucleotides of the invention by a chemical route.

The property of being able to “hybridize” onto polynucleotides means the ability of a polynucleotide or oligonucleotide to bind under stringent conditions to an almost complementary sequence, while there are no nonspecific bindings between noncomplementary partners under these conditions. For this purpose, the sequences should be 70-100%, preferably 90-100%, complementary. The property of complementary sequences being able to bind specifically to one another is made use of, for example, in the Northern or Southern blot technique or in PCR or RT-PCR in the case of primer binding. Oligonucleotides with a length of 30 base pairs or more are normally employed for this purpose. Stringent conditions mean, for example, in the Northern blot technique the use of a washing solution at 50-70° C., preferably 60-65° C., for example 0.1×SSC buffer with 0.1% SDS (20×SSC: 3M NaCl, 0.3M Na citrate, pH 7.0) for eluting nonspecifically hybridized cDNA probes or oligonucleotides. In this case, as mentioned above, only nucleic acids with a high degree of complementarity remain bound to one another.

These nucleic acids are preferably incorporated into expression constructs comprising, under the genetic control of regulatory nucleic acid sequences, a nucleic acid sequence coding for an enzyme of the invention; and vectors comprising at least one of these expression constructs. Such constructs of the invention preferably comprise a promoter 5′-upstream from the particular coding sequence, and a terminator sequence 3′-downstream, and, where appropriate, other usual regulatory elements, in particular each operatively linked to the coding sequence. “Operative linkage” means the sequential arrangement of promoter, coding sequence, terminator and, where appropriate, other regulatory elements in such a way that each of the regulatory elements is able to comply with its function as intended for expression of the coding sequence. Examples of sequences which can be operatively linked are targeting sequences and translation enhancers, polyadenylation signals and the like. Other regulatory elements comprise selectable markers, amplification signals, origins of replication and the like.

In addition to the artificial regulatory sequences it is possible for the natural regulatory sequences still to be present in front of the actual structural gene. This natural regulation can, where appropriate, be switched off by genetic modification, and expression of the genes can be increased or decreased. The gene construct may, however, also have a simpler structure, that is to say no additional regulatory signals are inserted in front of the structural gene, and the natural promoter with its regulation is not deleted. Instead, the natural regulatory sequence is mutated so that the regulation no longer takes place, and gene expression is enhanced or diminished. The nucleic acid sequences may be present in one or more copies in the gene construct.

Examples of usable promoters are: cos, tac, trp, tet, trp-tet, lpp, lac, lpp-lac, laclq, T7, T5, T3, gal, trc, ara, SP6, λ-PR or λ-PL promoter, which are advantageously used in gram-negative bacteria; and the gram-positive promoters amy and SPO2, the yeast promoters ADC1, MFα, AC, P-60, CYC1, GAPDH or the plant promoters CaMV/35S, SSU, OCS, lib4, usp, STLS1, B33, not or the ubiquitin or phaseolin promoter. The use of inducible promoters is particularly preferred, such as, for example, light- and, in particular, temperature-inducible promoters such as the PᵣPₗ promoter.

It is possible in principle for all natural promoters with their regulatory sequences to be used. In addition, it is also possible advantageously to use synthetic promoters.

Said regulatory sequences are intended to make specific expression of the nucleic acid sequences and protein expression possible. This may mean, for example, depending on the host organism, that the gene is expressed or overexpressed only after induction or that it is immediately expressed and/or overexpressed.

The regulatory sequences or factors may moreover preferably influence positively, and thus increase or reduce, expression. Thus, enhancement of the regulatory elements can take place advantageously at the level of transcription by using strong transcription signals such as promoters and/or enhancers. However, it is also possible to enhance translation by, for example, improving the stability of the mRNA.

An expression cassette is produced by fusing a suitable promoter to a suitable monooxygenase nucleotide sequence and to a terminator signal or polyadenylation signal. Conventional techniques of recombination and cloning are used for this purpose, as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector which makes optimal expression of the genes in the host possible. Vectors are well known to the skilled worker and can be found, for example, in “Cloning Vectors” (Pouwels P. H. et al., Eds., Elsevier, Amsterdam-New York-Oxford, 1985). Vectors mean not only plasmids but also all other vectors known to the skilled worker, such as, for example, phages, viruses such as SV40, CMV, baculovirus and adenovirus, transposons, IS elements, phasmids, cosmids, and linear or circular DNA. These vectors may undergo autonomous replication in the host organism or chromosomal replication.

Examples of suitable expression vectors which may be mentioned are:

Conventional fusion expression vectors such as pGEX (Pharmacia Biotech Inc; Smith, D. B. and Johnson, K. S. (1988) Gene 67:31-40), pMAL (New England Biolabs, Beverly, Mass.) and pRIT 5 (Pharmacia, Piscataway, N.J.), with which respectively glutathione S-transferase (GST), maltose E-binding protein and protein A are fused to the recombinant target protein.

Non-fusion protein expression vectors such as pTrc (Amann et al., (1988) Gene 69:301-315) and pET 11d (Studier et al. Gene Expression Technology: Methods in Enzymology 185, Academic Press, San Diego, Calif. (1990) 60-89).

Yeast expression vector for expression in the yeast S. cerevisiae, such as pYepSec1 (Baldari et al., (1987) Embo J. 6:229-234), pMFα (Kurjan und Herskowitz (1982) Cell 30:933-943), pJRY88 (Schultz et al. (1987) Gene 54:113-123) and pYES2 (Invitrogen Corporation, San Diego, Calif.). Vectors and processes for constructing vectors suitable for use in other fungi, such as filamentous fungi, comprise those which are described in detail in: van den Hondel, C. A. M. J. J. & Punt, P. J. (1991) “Gene transfer systems and vector development for filamentous fungi”, in: Applied Molecular Genetics of Fungi, J. F. Peberdy et al., Eds., p.1-28, Cambridge University Press: Cambridge.

Baculovirus vectors which are available for expression of proteins in cultured insect cells (for example Sf9 cells) comprise the pAc series (Smith et al., (1983) Mol. Cell Biol. 3:2156-2165) and the pVL series (Lucklow and Summers (1989) Virology 170:31-39).

Plant expression vectors such as those described in detail in: Becker, D., Kemper, E., Schell, J. and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721.

Mammalian expression vectors such as pCDM8 (Seed, B. (1987) Nature 329:840) and pMT2PC (Kaufman et al. (1987) EMBO J. 6:187-195).

Further suitable expression systems for prokaryotic and eukaryotic cells are described in Chapters 16 and 17 of Sambrook, J., Fritsch, E. F. and Maniatis, T., Molecular cloning: A Laboratory Manual, 2nd edition, Cold Spring Harbor Laboratory, Cold Spring Harbor Laboratory Press, Cold Spring Harbor, N.Y., 1989.

The expression constructs and vectors of the invention can be used to produce recombinant microorganisms which are transformed, for example, with at least one vector of the invention and can be employed for producing the enzymes used according to the invention and/or for carrying out the process of the invention. The recombinant constructs of the invention described above are advantageously introduced into a suitable host system and expressed. Cloning and transfection methods familiar to the skilled worker, such as, for example, coprecipitation, protoplast fusion, electroporation, retroviral transfection and the like, are preferably used to bring about expression of said nucleic acids in the particular expression system. Suitable systems are described, for example, in Current Protocols in Molecular Biology, F. Ausubel et al., Eds., Wiley Interscience, New York 1997.

Suitable host organisms are in principle all organisms which enable expression of the nucleic acids of the invention, their allelic variants, their functional equivalents or derivatives. Host organisms mean, for example, bacteria, fungi, yeasts, plant or animal cells. Preferred organisms are bacteria such as those of the genera Escherichia, such as, for example, Escherichia coli, Streptomyces, Bacillus or Pseudomonas, eukaryotic microorganisms such as Saccharomyces cerevisiae, Aspergillus, Blakeslea, Phycomyces, higher eukaryotic cells from animals or plants, for example Sf9 or CHO cells.

Successfully transformed organisms can be selected through marker genes which are likewise present in the vector or in the expression cassette. Examples of such marker genes are genes for antibiotic resistance and for enzymes which catalyze a color-forming reaction which causes staining of the transformed cell. These can then be selected by automatic cell sorting. Microorganisms which have been successfully transformed with a vector and have an appropriate antibiotic resistance gene (for example G418 or hygromycin) can be selected by appropriate antibiotic-containing media or nutrient media. Marker proteins presented on the cell surface can be used for selection by means of affinity chromatography.

The combination of the host organisms and the vectors appropriate for the organisms, such as plasmids, viruses or phages, such as, for example, plasmids with the RNA polymerase/promoter system, the phages λ or μ or other temperate phages or transposons and/or other advantageous regulatory sequences forms an expression system. The term “expression system” means, for example, the combination of mammalian cells, such as CHO cells, and vectors, such as pcDNA3neo vector, which are suitable for mammalian cells.

If required, the gene product can also be expressed in transgenic organisms such as transgenic animals such as, in particular, mice or sheep, or transgenic plants.

Recombinant production of the monooxygenases which can be employed according to the invention is also possible, in which case a monooxygenase-producing microorganism is cultivated, where appropriate expression of the monooxygenase is induced, and the monooxygenase is isolated from the culture. The monooxygenase can thus be produced on the industrial scale if this is desired.

The recombinant microorganism can be cultivated and fermented by known processes. Bacteria can be grown, for example, in TB or LB medium and at a temperature of 20 to 40° C. and at a pH of 6 to 9. Details of suitable culturing conditions are described, for example in T. Maniatis, E. F. Fritsch and J. Sambrook, Molecular Cloning: A Laboratory Manual, Cold Spring Harbor Laboratory, Cold Spring Harbor, N.Y. (1989).

If the monooxygenase is not secreted into the culture medium, the cells are then disrupted, and the enzyme is obtained from the lysate by known protein isolation methods. The cells may alternatively be disrupted by high-frequency ultrasound, by high pressure, such as, for example, in a French pressure cell, by osmolysis, by exposure to detergents, lytic enzymes or organic solvents, by homogenizers or by a combination of a plurality of the methods mentioned.

The monooxygenase can be purified by known chromatographic processes such as molecular sieve chromatography (gel filtration), such as Q-Sepharose chromatography, ion exchange chromatography and hydrophobic chromatography, and by other usual methods such as ultrafiltration, crystallization, salting out, dialysis and native gel electrophoresis. Suitable methods are described, for example, in Cooper, T. G., Biochemische Arbeitsmethoden, Verlag Walter de Gruyter, Berlin, New York or in Scopes, R., Protein Purification, Springer Verlag, New York, Heidelberg, Berlin.

It is particularly advantageous for isolation of the recombinant protein to use vector systems or oligonucleotides which extend the DNA by particular nucleotide sequences and thus code for modified polypeptides or fusion proteins which serve, for example, for simpler purification. Suitable modifications of this type are, for example, so-called tags which act as anchors, such as, for example, the modification known as hexahistidine anchor, or epitopes which can be recognized as antigens by antibodies (described, for example, in Harlow, E. and Lane, D., 1988, Antibodies: A Laboratory Manual. Cold Spring Harbor (N.Y.) Press). These anchors can be used to attach the proteins to a solid support, such as, for example, a polymer matrix, which can, for example, be packed into a chromatography column, or can be used on a microtiter plate or another support.

These anchors can at the same time also be used for recognition of the proteins. It is also possible to use for recognition of the proteins conventional markers such as fluorescent dyes, enzyme markers which form a detectable reaction product after reaction with a substrate, or radioactive labels, alone or in combination with the anchors for derivatizing the proteins.

The following nonlimiting examples describe specific embodiments of the invention.

### EXAMPLES

General Experimental Details:

a) General Cloning Methods

The cloning steps carried out for the purpose of the present invention, such as, for example, restriction cleavages, agarose gel electrophoresis, purification of DNA fragments, transfer of nucleic acids to nitrocellulose and nylon membranes, linkage of DNA fragments, transformation of E. coli cells, culturing of bacteria, replication of phages and sequence analysis of recombinant DNA, were carried out as described by Sambrook et al. (1989) loc. cit.

b) Polymerase Chain Reaction (PCR)

PCR was carried out in accordance with a standard protocol using the folowing standard mixture:

8 μl of dNTP mix (200 μM), 10 μl of Taq polymerase buffer (10×) without MgCl₂, 8 μl of MgCl₂ (25 mM), 1 μl of each primer (0.1 μM), 1 μl of DNA to be amplified, 2.5 U of Taq polymerase (MBI Fermentas, Vilnius, Lithuania), demineralized water ad 100 μl.

c) Cultivation of E. Coli

The recombinant E. coli DH5α strain was cultivated in LB-Amp medium (tryptone 10.0 g, NaCl 5.0 g, yeast extract 5.0 g, ampicillin 100 g/ml, H₂O ad 1000 ml) at 37° C. For this purpose, in each case one colony was transferred, using an inoculating loop, from an agar plate into 5 ml of LB-Amp. After cultivation for about 18 hours, shaking at a frequency of 220 rpm, 400 ml of medium in a 2 l flask were inoculated with 4 ml of culture. Induction of P450 expression in E. coli took place after the OD578 reached a value between 0.8 and 1.0 by heat-shock induction at 42° C. for three to four hours.

d) Cell Disruption

Cell pellets with a wet biomass of up to 15 g of E. coli DH5α were thawed on ice and suspended in 25 ml of potassium phosphate buffer (50 mM, pH 7.5, 1 mM EDTA) or Tris/HCl buffer (50 mM, pH 7.5, 1 mM EDTA). The ice-cooled E. coli cell suspension was disrupted by treatment with ultrasound (Branson Sonifier W250, (Dietzenbach, Germany), power output 80 W, working interval 20%) for three minutes. Before the protein purification, the cell suspension was centrifuged at 32 500 g for 20 min and filtered through a 0.22 mm Sterivex GP filter (Millipore), resulting in a crude extract.

### Example 1

Cloning and Expression of P450 from Thermus Thermophilus HB27 and the His tag Derivatives Thereof

1. Cloning of P450 from Thermus Thermophilus HB27

A clone (TTHB66) comprising the coding P450 sequence (also referred to as CYP175A1 gene hereinafter) was obtained from a Thermus gene library. The coding P450 sequence (blunt ended) was cloned into the HincII cleavage site of the plasmid pTZ19R (MBI Fermentas). The coding P450 sequence was amplified by a PCR from the plasmid TTHB66 obtained in this way. The following primers were used for this:
a) 30-mer sense oligonucleotide comprising the NdeI cleavage site (in italics) as part of the P450 ATG start codon: 5′-CGMGCTCATATGMGCGCCTTTCCCTGAG (SEQ ID NO:7).
b) 30-mer antisense oligonucleotide comprising the EcoRI cleavage site (in italics) as part of the TGA stop codon: 5′-GCGAATTCACGCCCGCACCTCCTCCCTAGG (SEQ ID NO:8).

The resulting fragment was cloned into the NdeI cleavage sites of the vector pCYTEXP1 (plasmid with the temperature-inducible P_{R}P_{L} promoter system of bacteriophage 8 (Belev T. N., et al., Plasmid (1991) 26:147)) and transformed into E. coli DH-5α (Clontech, Heidelberg).

i E. coli DH-5α comprising the plasmid of interest was inoculated into LB medium in the presence of ampicillin, and the culture was incubated at 37° C. overnight. Part of the sample was inoculated into fresh LB medium (in the presence of ampicillin), and the resulting culture was cultivated at 37° C. until the OD was 0.9. Induction took place by increasing the temperature to 42° C. over a period of 24 hours. The change in the P450 content during expression was determined on the basis of measurements of the CO difference spectrum.

| Expression time | | |
|---|---|---|
| [h] | ΔA₄₅₀₋₄₉₀ | P450 concentration [μM] |
| 4 | 0.092 | 0.056 |
| 8 | 0.176 | 0.106 |
| 24 | 0.106 | 0.064 |

2. Cloning of P450 from Thermus Thermophilus HB27 with N-terminal His tag

The coding P450 sequence was amplified by PCR from the plasmid TTHB66 using the following primers:
(a) 50-mer sense oligonucleotide comprising the NdeI cleavage site (in italics) as part of the P450 ATG start codon and the tag-encoding codons (underlined): 5′-CGMGCTCATATGCATCACCATCATCATCACAAGCGCCTTTC (SEQ ID NO:9);
(b) 30-mer antisense oligonucleotide comprising the EcoRI cleavage site (in italics) as part of the TGA stop codon: 5′-GCGAATTCACGCCCGCACCTCCTCCCTAGG (SEQ ID NO:8).

The resulting fragment was cloned into the NdeI and EcoRI cleavage sites of the vector p-CYTEXP1 and expressed in E. coli DH-5α.

E. coli DH-5α comprising the plasmid of interest was inoculated into LB medium in the presence of ampicillin, and the culture was incubated at 37° C. overnight. Part of the sample was inoculated into fresh LB medium (in the presence of ampicillin), and the resulting culture was cultivated at 37° C. until the OD was 0.9. Induction took place by increasing the temperature to 42° C. over a period of 24 hours. The change in the P450 content during expression was determined on the basis of measurements of the CO difference spectrum.

| Expression time | | |
|---|---|---|
| [h] | ΔA₄₅₀₋₄₉₀ | P450 concentration [μM] |
| 4 | ND | ND |
| 8 | 0.097 | 0.073 |
| 24 | 0.111 | 0.073 |

3. Cloning of P450 from Thermus Thermophilus HB27 with C-terminal His tag

The coding P450 sequence was amplified by PCR from the plasmid TTHB66 using the following primers:
(a) 30-mer sense oligonucleotide comprising the NdeI cleavage site (in italics) as part of the P450 ATG start codon: 5′-CGMGCTCATATGMGCGCCTTTCCCTGAG (SEQ ID NO:7)

(b) 47-mer antisense oligonucleotide comprising the EcoRi cleavage site (in italics) as part of the TGA stop codon and the underlined tag-encoding part-sequence:

| | |
|---|---|
| 5′-CG [symbol] AGTGATGATGATGGTGATGCGCC | (SEQ ID NO:10) |
| CGCACCTCCTC. | |

The resulting fragment was cloned into the NdeI and EcoRI cleavage sites of the vector p-CYTEXP1 and expressed in E. coli DH-5α.

E. coli DH-5α comprising the plasmid of interest was inoculated into LB medium in the presence of ampicillin, and the culture was incubated at 37° C. overnight. Part of the sample was inoculated into fresh LB medium (in the presence of ampicillin), and the resulting culture was cultivated at 37° C. until the OD was 0.9. Induction took place by increasing the temperature to 42° C. over a period of 24 hours. The change in the P450 content during expression was determined on the basis of measurements of the CO difference spectrum.

| Expression time | | P450 concentration |
|---|---|---|
| [h] | ΔA₄₅₀₋₄₉₀ | [μM] |
| 4 | ND | ND |
| 8 | 0.1 | 0.075 |
| 24 | ND | ND |

### Example 2

Determination of the Thermal Stability of P450 from Thermus Thermophilus Compared with P450 BM3

The two enzymes were each incubated in Tris/HCl buffer pH 7.5, 25 mM at various temperatures for 30 minutes. The mixtures were then cooled and the P450 concentration was determined by spectrometry. The results are summarized in the following table and shown as a graph in FIG. 2.

| | | | | | |
|---|---|---|---|---|---|
| Temperature [° C.] | | 30 | 40 | 50 | 60 |
| P450 concentration [%] | P450 thermus | 100 | 89 | 29 | 22 |
| | P450 BM3 | 92 | 63 | 0 | 0 |

As is evident from the experimental results, the enzyme of the invention has a significantly higher thermal stability after incubation at all the temperatures for 30 minutes.

### Example 3

Production of an Expression Vector for Cytochrome P450 Monooxygenase from T. Thermophilus HB 27

Plasmid DNA (clone TTHB66) comprising the coding sequence of cytochrome P450 monooxygenase (CYP175A1 gene) was the starting point. The polymerase chain reaction (PCR) was used to introduce EcoRI and PstI restriction cleavage sites into the CYP175A1 gene. The gene was amplified using the following primers:

| | |
|---|---|
| 5′CCGGAATTCATGAAGCGCCTTTCCCTGAGG; | (SEQ ID NO:11) |
| 5′CCAATGCATTGGTTCTGCAGTCAGGCCCGCA | (SEQ ID NO:12) |
| CCTCCTCCCTAGG | |

The new restriction cleavage sites are shown underlined. The reaction mixture for the PCR consisted of template DNA (100 ng), 2.5 U of pfu DNA polymerase (Stratagene), 5 μl of reaction buffer, 5 μl of DMSO, 0.4 μmol of each oligonucleotide, 400 μmol of dNTPs and H₂O ad 50 μl. The following PCR cycle parameters were set: 95° C., 1 minute; (95° C., 1 minute; 53° C., 1 minute 30 seconds; 68° C., 1 minute 30 seconds) 30 cycles; 68° C., 4 minutes. The CYP175A1 gene sequence was checked by DNA sequencing.

After restriction digestion of the PCR product, the CYP175A1 gene was cloned into the EcoRI and PstI cleavage sites of the plasmids pKK 223-3 (Amersham Pharmacia). pKK 223-3 contains the strong tac promoter upstream of a multiple cloning site and the strong rrnB ribosomale terminator downstream thereof to control protein expression. The resulting plasmid is called pKK_CYP.

### Example 4

Biotransformation of β-Carotene in Recombinant E. Coli Strains

Recombinant E. coli strains capable, through heterologous complementation, of producing β-carotene were produced for the β-carotene biotransformation.

Strains of E. coli JM109 were used as host cells for the complementation experiments with the plasmids pACYC_Y and pKK_CYP (prepared as in Example 3). The plasmid pACYC_Y harbors the carotenogenic genes crtE, crtB, crtlC14 and crtY, isolated from E. uredovora. Said genes were in each case cloned in with their own lac promoter in order to make expression possible. Production of these plasmids is described in the thesis by I. Kauffmann, Erhöhung mikrobieller Diversität von Carotinoiden, June 2002, Institute of Technical Biology, Stuttgart University. The precursor construct comprising the carotenogenic genes crtE, crtB, crtlC14 is described in Schmidt-Dannert (2000), Curr. Opin. Biotechnol. 11, 255-261.

Further details of the heterologous complementation are also described, for example, in Ruther, A. Appl. Mikrobiol. Biotechnol. (1997) 48: 162-167; Sandmann, G., Trends in Plant Science (2001) 6: 1, 14-17 and Sandmann, G. et al., TIBTECH (1999), 17: 233-237.

The disclosure in the abovementioned publications is hereby incorporated by reference.

Cultures of E. coli JM109 were transformed in a manner known per se with the plasmids pACYC_Y and pKK_CYP and cultivated in LB medium at 30° C. and 37° C. for two days. Ampicillin (1 μg/ml), chloramphenicol (50 μg/ml) and isopropyl β-thiogalactoside (1 mmol) were added in a conventional way. As a comparison sample, an E. coli JM109 strain was transformed only with the plasmid pACYC_Y and cultivated in the same way.

The carotenoids were isolated from the recombinant E. coli strains by extraction of the cells with acetone and then with hexane. The combined extracts were partitioned with water. The organic phase was isolated, evaporated to dryness and fractionated by HPLC on a DXSIL C8 column with water/acetonitrile (5:95). The following conditions were set for the process:

Separating column: DXSIL C8, 3 μm, 120 A, 2.1×100 mm

Flowrate: 0.35 mL/min

Eluent: isocratic water/acetonitrile 5/95

Detection:
UV_VIS_{—}1 st wavelength=453 nm
UV_VIS_{—}1st bandwidth=4 nm
3DFIELD.Max. wavelength=600 nm
3DFIELD.Min. wavelength=190 nm
3DFIELD.Ref. wavelength=399 nm
3DFIELD.Ref. bandwidth=40 nm

The spectra were determined directly from the eluted peaks using a diode array detector. The isolated substances were identified via their absorption spectra and their retention times by comparison with standard samples.

Chromatograms of the standards for β-carotene, zeaxanthin and cryptoxanthin are shown in the appended FIGS. 4A to 4C. FIG. 5A shows the chromatographic analysis of a sample obtained from the E. coli strain transformed with the pACYC_Y plasmid. It is evident that the latter is able to produce β-carotene owing to the heterologous complementation. FIG. 5B shows the chromatogram of a E. coli strain produced with heterologous complementation according to the invention and additionally transformed with the pKK_CYP plasmid. It is surprisingly evident in this case that, besides β-carotene, there are significant amounts of the corresponding hydroxylation products zeaxanthin and cryptoxanthin detectable.

1. A process for the oxidation of carotenoids, which comprises reacting a carotenoid in the presence of an enzyme having cytochrome P450 monooxygenase activity from bacteria of the genus Thermus sp., and isolating the oxidation product.

2. A process as claimed in claim 1, wherein
a1) a recombinant microorganism which produces an enzyme having cytochrome P450 monooxygenase activity is cultivated in a culture medium in the presence of exogenous β-carotene or β-carotene formed as intermediate; or
a2) a β-carotene-containing reaction medium is incubated with an enzyme having cytochrome P450 monooxygenase activity; and
b) the oxidation product formed or a secondary product thereof is isolated from the medium.

3. A process as claimed in claim 2, wherein the oxidation product comprises zeaxanthin, cryptoxanthin, adonirubin, astaxanthin, lutein or mixtures thereof.

4. A process as claimed in claim 2, wherein the oxidation is carried out by cultivating the microorganism in the presence of oxygen at a cultivation temperature of at least about 20° C. and at a pH of about 6 to 9.

5. A process as claimed in claim 4, wherein the microorganism is able, through heterologous complementation, to produce carotenoids and additionally expresses an enzyme having cytochrome P450 monooxygenase activity.

6. A process as claimed in claim 1, wherein the carotenoid is added as exogenous substrate to a medium, and the oxidation is carried out by enzymatic reaction of the substrate-containing medium in the presence of oxygen at a temperature of at least about 20° C. and at a pH of about 6 to 9.

7. A process as claimed in claim 1, wherein the cytochrome P450 monooxygenase has an amino acid sequence which comprises a part-sequence from amino acid residue Pro328 to Glu345 as shown in SEQ ID NO:2.

8. A process as claimed in claim 1, wherein the cytochrome P450 monooxygenase has an amino acid sequence which comprises a part-sequence from amino acid residue Val216 to Ala227 as shown in SEQ ID NO:2.

9. A process as claimed in claim 1, wherein the cytochrome P450 monooxygenase has an amino acid sequence which comprises at least one part-sequence which is selected from part-sequences of at least 10 consecutive amino acids from the sequence regions specified by the amino acid residues Met1 to Phe327 and Gly346 to Ala389 as shown in SEQ ID NO:2.

10. A process as claimed in claim 1, wherein the cytochrome P450 monooxygenase has an amino acid sequence which essentially corresponds to SEQ ID NO:2.

11. A process as claimed in claim 1, wherein the cytochrome P450 monooxygenase is from a bacterium of the species Thermus thermophilus used.

12. A process as claimed in claim 2, where a recombinant microorganism which harbors an expression construct which comprises, under the control of regulatory nucleotide sequences, the coding sequence for a cytochrome P450 monooxygenase is cultivated, wherein the cytochrome P450 monooxygenase has an amino acid sequence
(a) which comprises a part-sequence from amino acid residue Pro 328 to Glu345 as shown in SEQ ID NO:2; and/or
(b) which comprises a part-sequence from amino acid residue Val216 to Ala227 as shown in SEQ ID NO:2; and/or
(c) which comprises at least one part-sequence which is selected from part-sequences of at least 10 consecutive amino acids from the sequence regions specified by the amino acid residues Met1 to Phe327 and Gly345 to Ala389 as shown in SEQ ID NO:2; and/or
(d) which essentially corresponds to SEQ ID NO:2; and/or
(e) which is from a bacterium of the species Thermus thermophilus.

13. (canceled)

14. A recombinant microorganism which is able through heterologous complementation to produce β-carotene and additionally expresses an enzyme having cytochrome P450 monooxygenase activity.

15. A microorganism as claimed in claim 14, in which the heterologous complementation is with carotenogenic genes.

16. A microorganism as claimed in claim 14, derived from bacteria of the genus Escherichia sp.

17. A microorganism as claimed in claim 16, derived from E. coli.

18. A microorganism as claimed in claim 14, transformed with an expression vector which comprises, under the genetic control of regulatory nucleotide sequences, the coding sequence for a cytochrome P450 monooxygenase.

19. An expression vector comprising the coding sequence for a cytochrome P450 monooxygenase as defined in any of claims 7 to 11, which is operatively linked upstream to the strong tac promoter and downstream to the strong rm B ribosomal terminator, wherein the cytochrome P450 monooxygenase has an amino acid sequence
(a) which comprises a part-sequence from amino acid residue Pro 328 to Glu345 as shown in SEQ ID NO:2; and/or
(b) which comprises a part-sequence from amino acid residue Val216 to Ala227 as shown in SEQ ID NO:2; and/or
(c) which comprises at least one part-sequence which is selected from part-sequences of at least 10 consecutive amino acids from the sequence regions specified by the amino acid residues MetI to Phe327 and Gly345 to Ala389 as shown in SEQ ID NO:2; and/or
(d) which essentially corresponds to SEQ ID NO:2; and/or
(e) which is from a bacterium of the species Thermus thermophilus.

20. A process of claim 6, wherein the substrate-containing medium further comprises about 10- to 100-fold molar excess, based on the substrate, of reducing equivalents.

21. A microorganism as claimed in claim 17 derived from E. Coli MJ 109.
